(19) United States
(12) Patent Application Publication
(10) Pub. No.: US 2024/0132920 A1
(43) Pub. Date: Apr. 25, 2024

(54) PREFLASH SYSTEM AND METHOD

(71) Applicant: IntegroEnergy Technologies LLC, Lake Crystal, MN (US)

(72) Inventors: Daniel W. Sonnek, Lake Crystal, MN (US); Kyle Matthew Sieh, Mapleton, MN (US)

(21) Appl. No.: 18/489,953

(22) Filed: Oct. 18, 2023

Related U.S. Application Data
(60) Provisional application No. 63/380,353, filed on Oct. 20, 2022.

(51) Int. Cl. C12P 7/06; B01D 3/00

(52) U.S. Cl. CPC C12P 7/06; B01D 3/001

(57) ABSTRACT

The disclosure relates to a system and method for preflash processing used in dry mill ethanol facilities, including routing beer to a baffled flash tank, in which the beer is processed and then separated into a vapor stream and liquid beer. Preheating the beer via preflash processing efficiently recovers energy within the system, thereby reducing the need for additional energy input.

### CROSS-REFERENCE TO RELATED APPLICATIONS

This application claims priority to U.S. Provisional Patent Application No. 63/380,353 filed Oct. 20, 2022, the disclosure of which is hereby incorporated by reference in its entirety.

### FIELD

This disclosure pertains to a method and system for preflash processing used in dry mill ethanol facilities, including routing beer to a baffled flash tank, in which the beer is processed and then separated into a vapor stream and liquid beer before further processing in a distillation column.

### BACKGROUND

Standard dry mill ethanol facilities have been designed to grind whole corn or similar grain to fine flour, hydrate, heat, expose to enzymes, and then hold at a relatively high temperature to convert the grain starches into sugars. This holding step is called liquefaction. After liquefaction, the resulting mash, which is hot, is sent through a fermentation process, where yeast converts the sugars to ethanol. After this, the fermented mixture (called “beer”) is sent to a distillation system comprising one or more beer distillation columns and rectifiers to extract the ethanol for concentration and sale. In order to minimize energy input, heat recovery systems are often found in these plants. For instance, the hot mash directed from liquefaction to fermentation must be cooled, e.g., from about 190° F. down to about 90° F., to allow the yeast to survive and flourish in the fermenters. Much of this heat is transferred to the beer that is being routed to distillation. The remaining heat is removed via trim cooling and wasted by transfer to a cooling system.

Thus, there is a need for a system and method for improving the energy efficiency of the plant, in particular, with respect to the use of the heat recovery system and decrease of heat waste.

### SUMMARY

A method of preflash processing in a dry mill ethanol plant is disclosed. The method includes: (a) routing warm mash to a heat recovery system to produce cold mash; (b) fermenting the cold mash to produce a cold beer stream; (c) routing the cold beer stream into the heat recovery system, wherein the heat recovery system is configured to transfer heat between the warm mash and the cold beer stream resulting in the cold mash and warm beer stream; (d) routing the warm beer stream to a baffled flash tank, wherein the baffled flash tank is configured to separate the warm beer stream into a first vapor stream and liquid beer; (e) routing the liquid beer into a beer distillation column, wherein the beer distillation column is configured to separate the liquid beer into a second vapor stream and solid precipitates; and (f) discharging the first vapor stream from the baffled flash tank into a beer distillation column vapor discharge line.

In various embodiments, the method may further comprise discharging the second vapor stream from the beer distillation column by the beer distillation column vapor discharge line. In other embodiments, the method may further comprise combining the second vapor stream from the beer distillation column with the first vapor stream from the baffled flash tank. In another embodiment, prior to the routing in step (c), the method may further comprise pressurizing the cold beer stream to a range of about 55 psia to about 115 psia.

In various embodiments, the warm mash may have a temperature in a range of about 180° F. to about 200° F. In various embodiments, the cold mash may have a temperature in a range of about 110° F. to about 130° F. In some embodiments, the cold beer stream may have a temperature in a range of about 80° F. to about 95° F. and may comprise an ethanol concentration in a range of about 5% to about 30% v/v. In other embodiments, the warm beer stream may have a temperature in a range of about 155° F. to about 170° F. and may comprise an ethanol concentration in a range of about 5% to about 30% v/v.

In various embodiments, the liquid beer may have a temperature in a range of about 150° F. to about 160° F. and may comprise an ethanol concentration in a range of about 5% to about 30% v/v. In various embodiments, the first vapor stream may comprise an ethanol concentration in a range of about 50% to about 60% v/v. In other embodiments, the second vapor stream may comprise an ethanol concentration in a range of about 50% to about 60% v/v.

In various embodiments, the baffled flash tank may be coupled to the beer distillation column by two outlets such that pressure and temperature of the baffled flash tank and the top of the beer distillation column are substantially equivalent; wherein a first outlet is configured to route the first vapor stream from the baffled flash tank to the beer distillation column vapor discharge line; and wherein a second outlet is configured to route the liquid beer from the baffled flash tank to the beer distillation column.

In various embodiments, the pressure of the baffled flash tank and beer distillation column may be in a range of about 3 psia to about 20 psia. In various embodiments, the temperature of the baffled flash tank and the top of the beer distillation column may be in a range of about 155° F. to about 165° F.

A system for a dry mill ethanol plant is disclosed comprising: a heat recovery system coupled to a baffled flash tank, wherein the baffled flash tank may be configured to receive a warm beer stream; and a distillation system coupled to the baffled flash tank, wherein the baffled flash tank may be configured to separate the warm beer stream into a vapor stream and liquid beer. In various embodiments, the baffled flash tank may comprise two outlets, a first outlet coupled to a beer distillation column vapor discharge line for routing the vapor stream from the baffled flash tank; and a second outlet coupled to a beer distillation column inlet for routing the liquid beer from the baffled flash tank.

### BRIEF DESCRIPTION OF THE DRAWINGS

FIG. 1 is a schematic that depicts an example of a system and method of the present disclosure.

FIG. 2A is a schematic that depicts an example of a heat recovery system of the present disclosure.

FIG. 2B is a schematic that depicts another example of a heat recovery system of the present disclosure.

### DETAILED DESCRIPTION

The following discussion omits or only briefly describes conventional features of the disclosed technology that are apparent to those skilled in the art. Reference to various embodiments does not limit the scope of the claims attached hereto. Additionally, any examples set forth in this specification are intended to be non-limiting and merely set forth some of the many possible embodiments for the appended claims. Further, particular features described herein can be used in combination with other described features in each of the various possible combinations and permutations. A person of ordinary skill in the art would know how to make and use the disclosed technology, in combination with routine experiments, to achieve other outcomes not specifically disclosed in the examples or the embodiments.

Unless otherwise specifically defined herein, all terms are to be given their broadest possible interpretation including meanings implied from the specification as well as meanings understood by those skilled in the art and/or as defined in dictionaries, treatises, etc. Unless defined otherwise, all technical and scientific terms used herein have the same meaning as commonly understood by one of ordinary skill in the art in the field of the disclosed technology. It must also be noted that, as used in the specification and the appended claims, the singular forms “a,” “an” and “the” include plural referents unless otherwise specified, and that the terms “includes” and/or “including,” when used in this specification, specify the presence of stated features, elements, and/or components, but do not preclude the presence or addition of one or more other features, steps, operations, elements, components, and/or groups thereof. Additionally, methods, equipment, and materials similar or equivalent to those described herein can also be used in the practice or testing of the disclosed technology.

As used herein, the term “about” with respect to a numerical value means plus or minus 10% of the numerical value, unless indicated otherwise.

As used herein, various terms are used such as “first,” “second,” and the like. These terms are words of convenience in order to distinguish between different elements, and such terms are not intended to be limiting as to how the different elements may be utilized.

The systems disclosed herein include a baffled flash tank for preflash processing of beer, which is routed to a distillation system. The beer preflash processing was developed and designed to recover most of the energy in the heat recovery equipment and allow the beer temperature to be self-limiting. As the baffled flash tank is directly coupled to the beer distillation column at more than one location, i.e., via a liquid outlet and a vapor outlet, the pressure of the baffled flash tank and top of the beer distillation column will be about the same. Because the pressures are identical, the warm beer that enters the baffled flash tank will flash to vapors in order to cool the remaining liquid, and the cooled liquid will be about the same temperature as the top of the beer distillation column.

As used herein, the term “flash” or “flashing” refers to a process of liquid that undergoes a rapid phase change from a liquid phase to a vapor phase when exposed to specific conditions, such as a decrease in pressure or an increase in temperature. As used in this document, the term “preflashing” (or “preflash”) is intended to mean flashing the warm beer stream in a baffled flash tank before routing it to a distillation system.

Preheating the beer via preflash processing saves a significant amount of energy, e.g., the energy required to raise the temperature of the liquid beer to produce vapors for distillation, as the ethanol in the beer stream entering the beer distillation column is at a flash point. This ensures that the beer stream entering the beer distillation column does not need a significant amount of additional energy input to raise the beer temperature to a flash point. Essentially, all of the energy injected into the beer distillation column from the baffled flash tank will be utilized to extract ethanol from the beer stream. Thus, less energy is required to move the ethanol production to completion.

In standard dry mill ethanol facilities, mash fermentation is achieved at a significantly lower temperature than liquefaction, which produces warm mash, the primary substance used in fermentation. Thus, a heat recovery system such as heat exchangers and/or trim coolers is utilized to lower the mash temperature such that it is suitable for fermentation. Accordingly, heat exchangers are used to recover some of this heat from the mash and transfer it to a cold beer stream, thus raising the beer temperature close to the distillation temperature. However, this heat transfer process must be carefully monitored in order not to overheat the beer. For instance, if the heat recovery equipment is configured to transfer as much heat as possible, the temperature of the beer will be significantly above the flash point, which is unsuitable for distillation. This can lead to piping and equipment damage through a water hammer resulting from an abrupt pressure change in the column lines between the fermenter and the beer distillation column. Moreover, the beer that is too hot can also have an adverse effect on beer distillation column carryover. This phenomenon occurs when the vapor loading at the top of the beer distillation column is at a state that retains solids. This can cause fouling downstream in the subsequent distillation, rectification, and dehydration steps, resulting in solids carrying over into the rectifier that can foul the rectifier trays and packing, resulting in lower efficiency distillation and even plant outages requiring cleaning. As such, a limited heat transfer from the warm mash to beer is possible and the rest of the heat is wasted/released to the cooling system, such as the trim cooler. This reduces the plant's overall efficiency and can unnecessarily load the plant's cooling utilities.

The presently disclosed technology is designed to optimize heat recovery while preventing the adverse side effects of beer overheating, thus reducing the overall energy requirement in the plant. The disclosed method includes: routing warm mash to a heat recovery system to produce cold mash; fermenting the cold mash to produce a cold beer stream; routing the cold beer stream into the heat recovery system, wherein the heat recovery system is configured to transfer heat between the warm mash and the cold beer stream resulting in the cold mash and warm beer stream; routing the warm beer stream to a baffled flash tank, wherein the baffled flash tank is configured to separate the warm beer stream into a first vapor stream and liquid beer routing the liquid beer into a beer distillation column, wherein the beer distillation column is configured to separate the liquid beer into a second vapor stream and solid precipitates; and discharging the first vapor stream from the baffled flash tank into a beer distillation column vapor discharge line.

The warm mash is produced by any known liquefaction processing in the art. The temperature of the warm mash before entering the heat recovery system may be in a range of about 180° F. to about 200° F., such as about 185° F. to about 195° F., or at about 190° F. After routing through the heat recovery system, the warm mash may be cooled to a temperature in a range of about 110° F. to about 130° F., such as about 115° F. to about 125° F., or at about 120° F., and now referred to as cold mash. The cold mash may be routed through a trim cooler for additional cooling before being routed for fermentation. Any known trim cooler and means for fermentation in the art may be used in accordance with this disclosure.

The fermentation of the cold mash produces a cold beer stream with a relatively low temperature, for example, in a range of about 80° F. to about 95° F., such as about 83° F. to about 92° F., or about 85° F. to about 90° F. In various embodiments, the cold beer stream may comprise a low ethanol concentration, for example, in a range of about 5% to about 30%, such as about 10% to about 27%, or about 12% to about 20% v/v.

The cold beer stream may be pumped and optionally pressurized to a range of about 55 psia to about 115 psia, such as about 65 psia to about 105 psia, about 75 psia to about 95 psia, or about 80 psia to about 85 psia, to provide the motive force to route through the heat recovery system where heat is transferred between the warm mash and the cold beer stream. The transferred heat may result in warm beer stream and cold mash. In various embodiments, the resulting warm beer stream may comprise a low ethanol concentration, for example, in a range of about 5% to about 30%, such as about 10% to about 27%, or about 12% to about 20% v/v. Moreover, the warm beer stream may have a temperature in a range of about 155° F. to about 170° F., such as about 158° F. to about 167° F., or at about 162° F., above the flash point of the beer, e.g., which is in a range about 150° F. to about 160° F. depending on the pressure of the beer distillation column. In various embodiments, the maximum beer temperature may be unrestricted, so the heat recovery system may be optimized for maximum heat exchange.

Because the warm beer stream is processed in the baffled flash tank before it is routed to a beer distillation column, the restrictive upper-temperature limit constraint of the conventional system is overcome. Notably, almost complete heat transfer between the warm mash and cold beer stream in the heat recovery system is achieved, and no measurable heat is wasted to the trim cooler or the environment. The presently disclosed system and process allow for a maximized cooling of the warm mash for fermentation while simultaneously transferring the heat energy to the cold beer stream, thus requiring less energy input for additional cooling of the warm mash (for fermentation) and heating of the warm beer stream (for distillation). In various embodiments, more than one expandable heat exchanger may be configured to operate in parallel to increase the heat transfer area between the warm mash and cold beer stream.

The heat recovery system is coupled to a baffled flash tank by any means known in the art, for example, by a pump and piping system. The baffled flash tank provides preflash processing of the warm beer stream before routing it to the beer distillation column. This way, the warm beer stream may be separated into vapor and liquid beer for energy-efficient use in the distillation process. In various embodiments, the baffled flash tank may comprise one or more baffles or plates in the interior of the flash tank that are configured to create obstructions in the flow of the warm beer stream and increase the surface area through which the warm beer travels. In various embodiments, the temperature of the liquid beer may be in a range of about 150° F. to about 160° F., such as about 153° F. to about 157° F., or at about 155° F. In various embodiments, the liquid beer may comprise a low ethanol concentration, for example, in a range of about 5% to about 30%, such as about 7% to about 25%, or about 10% to about 18% v/v.

As the velocity of the warm beer stream is impeded by the baffles in the flash tank, the warm beer stream undergoes a drop in pressure, causing the separation of a vapor stream comprising ethanol and water from the liquid beer comprising solid precipitates. Moreover, the baffles also create a path for said vapor stream that prevents any solid precipitates from being carried with the vapor stream. In various embodiments, the ethanol concentration of this vapor stream may be in a range of about 50% to about 60% v/v, such as about 50% to about 55% v/v, or about 55% to about 60% v/v.

In various embodiments, the baffled flash tank may be configured to receive the warm, optionally pressurized beer from a preceding vessel (e.g., fermenter or heat exchanger). In various embodiments, the warm beer stream may be pressurized to a range of about 65 psia to about 90 psia, such as about 70 psia to about 85 psia, or about 75 psia to about 80 psia. The baffled flash tank receives this warm, optionally pressurized beer stream and exposes it to a lower temperature of the baffled flash tank, the baffled flash tank is operated in a temperature range from about 155° F. to about 165° F., or at about 160° F. This temperature drop is accompanied by a drop in pressure, which causes the flashing of some of the warm beer stream in order to equilibrate the temperature gradient and the pressure in the baffled flash tank. This flashing process separates warm beer stream into a vapor stream and liquid beer.

In various embodiments, the baffled flash tank may comprise more than one outlet connected to a beer distillation column. In various embodiments, a first outlet, e.g., a vapor outlet, of the baffled flash tank may be connected to a vapor discharge line of the beer distillation column. This connection provides the discharged vaporous contents from the beer distillation column and the baffled flash tank to combine at a junction in the lines before being routed to a rectifier for additional processing. In various embodiments, a second outlet, e.g., a liquid outlet, of the baffled flash tank may be coupled to an inlet on the beer distillation column to route the liquid beer from the baffled flash tank to the beer distillation column. By connecting the baffled flash tank to both an inlet and an outlet of the beer distillation column such that it creates a circular system, the temperature of the liquid beer that is being routed from the baffled flash tank to the beer distillation column is ensured to be substantially equivalent to (within about 5° F.) or the same as the temperature at the top of the beer distillation column, e.g., in a range of about 155° F. to about 165° F., or at about 160° F., since the pressures between the two vessels are substantially equivalent (within about 0.2 psia) or the same, e.g., in a range of about 3 psia to about 20 psia, such as about 5 psia to about 15 psia, or about 8 psia to about 12 psia.

In various embodiments, the second outlet of the baffled flash tank may be configured to route the liquid beer from the liquid outlet of the baffled flash tank to the inlet of the beer distillation column. The liquid beer is then separated into a vapor stream of ethanol and water, which is routed to the top of the beer distillation column, and residual solid precipitates from processed grains, which are settled at the bottom of the beer distillation column. In various embodiments, the ethanol concentration of this vapor stream may be in a range of about 50% to about 60% v/v, such as about 50% to about 55% v/v, or about 55% to about 60% v/v. This vapor stream is released from the top of the beer distillation column via the vapor discharge line, where it combines with the vapor stream from the vapor outlet of the baffled flash tank. The combined vapor stream is then routed for rectification and dehydration to raise the ethanol concentration of the combined vapor stream and produce pure ethanol.

By introducing preflash processing in the baffled flash tank before distillation, the methods and systems disclosed herein prevent the adverse effects of temperature, pressure, and flow variations that can occur in the conventional process. In addition, the methods and systems disclosed herein achieves maximum heat recovery from the warm mash by preventing energy loss into a cooling system, but instead transferring it to the cold beer stream. By maximizing the heat recovery, less energy is required to be added to the system for distillation processing.

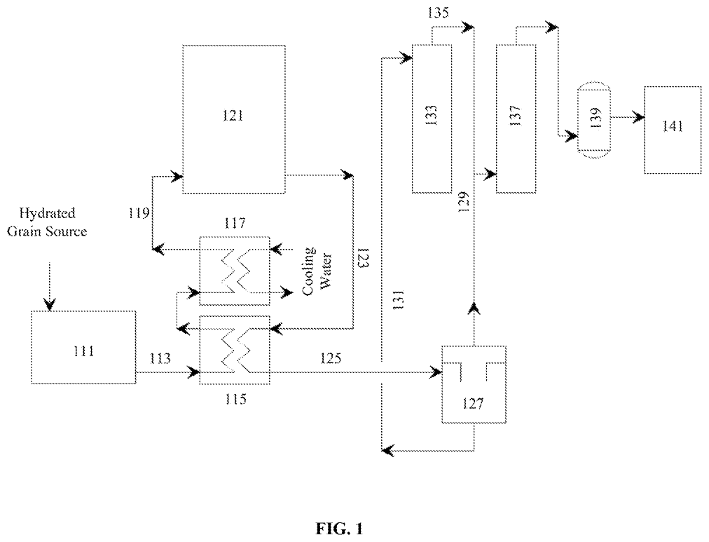

A system for a dry mill ethanol plant is also disclosed comprising a heat recovery system coupled to a baffled flash tank and a distillation system coupled to said baffled flash tank. Referring now to FIG. 1, a system and process of the disclosure are shown. The liquefaction of the hydrated grain source in reactor 111 produces a warm mash. This warm mash is then routed through heat exchanger 115, by line 113 where heat from the warm mash is transferred to cold beer stream 123 on the opposite side of heat exchanger 115. The warm mash is then routed through water-cooled trim cooler 117 to further lower the temperature to produce a cold mash. The cold mash is then routed to fermenter 121 by line 119 where it is converted to cold beer stream 123. Cold beer stream 123 is routed through heat exchanger 115 producing warm beer stream 125. Warm beer stream 125, which is above the flash point of alcohol in the beer, is then discharged into baffled flash tank 127 where an ethanol-containing vapor stream and liquid beer are produced. This first vapor stream is discharged from baffled flash tank 127 by vapor outlet 129, while the liquid beer is routed to beer distillation column 133 by liquid outlet 131, where a second ethanol-containing vapor stream is produced from the liquid beer. This second vapor stream is released from the top of beer distillation column 133 by vapor discharge line 135 and combines with the first vapor stream from vapor outlet 129. The combined vapor stream is then routed to rectifier 137 and dehydration system 139 to raise the ethanol concentration to a maximum, i.e., about 100% v/v ethanol. The resulting pure ethanol is then routed to ethanol storage 141 for packaging and sale.

### EXAMPLE

The present invention is next described by means of the following example. The use of these and other examples anywhere in the specification is illustrative only, and in no way limits the scope and meaning of the invention or of any exemplified form. Likewise, the invention is not limited to any particular preferred embodiments described herein. Indeed, modifications and variations of the invention may be apparent to those skilled in the art upon reading this specification and can be made without departing from its spirit and scope. The invention is therefore to be limited only by the terms of the claims, along with the full scope of equivalents to which the claims are entitled.

### Example 1

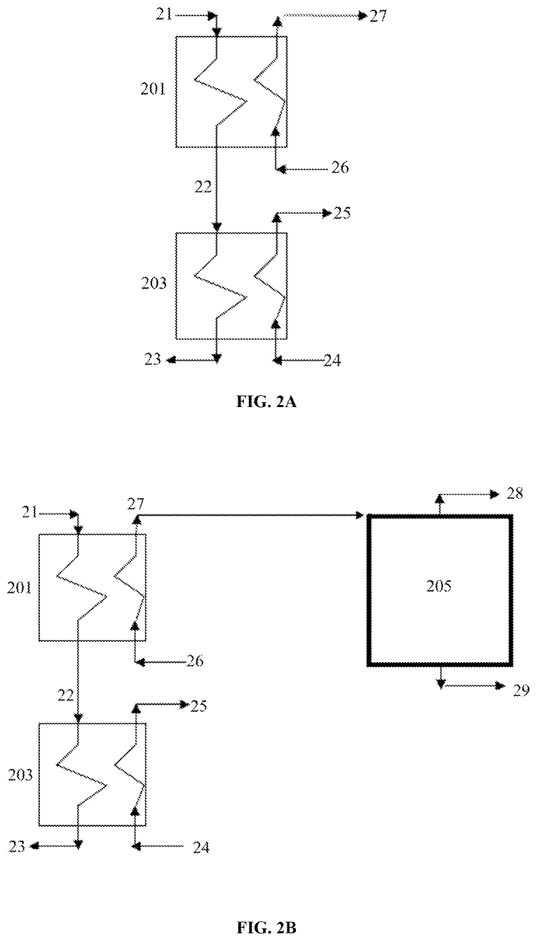

This example relates to beer feed overdrive mass and energy balance between a standard, non-preflash system and the disclosed preflash system. FIG. 2A depicts a standard Beer-Mash heat exchange system without preflash processing. As shown in FIG. 2A, warm mash 21 is routed through heat exchanger 201, transferring heat to an equivalent amount of beer 26 flowing in the opposite direction in heat exchanger 201. After being heated by warm mash 21, discharged beer 27 is at a higher temperature and routed for distillation. Conversely, exiting mash 22, now at a lower temperature, enters trim cooler 203, which lowers the temperature further, producing cold mash 23. Trim cooler 203 is operated by flowing a suitable amount of cooling water 24 to cool the mash in trim cooler 203. Discharged water 25 is at a higher temperature than cooling water 24 due to heat transfer from the mash stream in trim cooler 203.

Now referring to FIG. 2B, a Beer-Mash heat exchange system with preflash processing is shown. In FIG. 2B, baffled flash tank 205 coupled to heat exchanger 201 provides preflash processing of discharged beer 27. Discharged beer stream 27 enters baffled flash tank 205 and separates into vapor 28 and liquid beer 29. Vapor 28 is routed to rectification, while liquid beer 29 is routed to distillation. By incorporating baffled flash tank 205, the heat/temperature profiles of the mash, beer, and cooling water and the system's energy efficiency are significantly altered, as further explained below.

The efficiency of the disclosed preflash system and a standard, non-preflash system was investigated by monitoring the mash and beer temperatures at various points during the heat transfer process. Tables 1 and 2 summarize the temperature profiles of the standard and the preflash systems. In both systems, heat transfer between the warm mash and cold beer in the heat exchanger produces lower-temperature mash for fermentation and warm beer, as shown in FIGS. 2A and 2B. The starting temperature of the warm mash was measured at 185° F. in both systems before entering the heat exchanger. In the heat exchanger, heat transfer between the warm mash and cold beer lowered the warm mash temperature and raised the beer temperature.

In the standard system, the mash temperature was lowered from 185° F. to 130° F., compared to 119° F. in the preflash system. After being discharged from the heat exchanger, the mash from both systems was routed through a trim cooler to further lower the temperature to 92° F. for fermentation. The trim cooler utilized a cooled water stream measured at 75° F. as the external cooling source. The standard system required 4,000 gallons per minute (gpm) of water flow to cool the mash from 130° F. to 92° F., requiring 21.1 Millions of British Thermal Units per hour (MMBTU/hr) of energy to operate the trim cooler. In the preflash system, 37.5% less water flow, i.e., 2,500 gpm, was required to achieve the same cooling effect, requiring only 13.2 MMBTU/hr of energy to operate the trim cooler, a 37% reduction in the energy requirement.

TABLE 1
Temperature profiles of mash and beer in a standard,
non-preflash heat recovery system

| | Temp. before | Temp. after | Temp. after |
|---|---|---|---|
| | heat exchanger (° F.) | heat exchanger (° F.) | trim cooler (° F.) |
| Mash | 185 | 130 | 92 |
| Beer | 90 | 155 | N/A |

TABLE 2
Temperature profiles of mash and beer
in a preflash heat recovery system

| | Temp. before | Temp. after | Temp. after |
|---|---|---|---|
| | heat exchanger (° F.) | heat exchanger (° F.) | trim cooler (° F.) |
| Mash | 185 | 119 | 92 |
| Beer | 90 | 165 | N/A |

Conclusions: Overall, the disclosed preflash system achieves maximum heat transfer between the warm mash and the cold beer, reducing the plant's energy requirement by 37% in the heat exchange/heat recovery process. Additionally, compared to the standard system, which limits the maximum beer temperature to 155° F. to prevent temperature-induced fouling downstream, the disclosed preflash system promotes higher beer temperature, e.g., 165° F., to drive the preflash processing of some of the beer in the baffled flash tank before being routed to the beer distillation column, thus preventing any temperature, pressure, flow variation induced side-effects. The disclosed process results in an overall improved distillation efficiency and reduced plant energy requirement.

The foregoing merely illustrates the principles of the disclosure. Any examples set forth in this specification are not intended to be limiting and merely set forth some of the many possible embodiments for the appended claims. Those skilled in the art will readily recognize various modifications and changes that may be made without following the example embodiments and applications illustrated and described herein, and without departing from the true spirit and scope of the following claims.

All references cited and/or discussed in this specification are incorporated herein by reference in their entireties and to the same extent as if each reference was individually incorporated by reference.

We claim:

1. A method of preflash processing in a dry mill ethanol plant comprising:
(a) routing warm mash to a heat recovery system to produce cold mash;
(b) fermenting the cold mash to produce a cold beer stream;
(c) routing the cold beer stream into the heat recovery system, wherein the heat recovery system is configured to transfer heat between the warm mash and the cold beer stream resulting in the cold mash and warm beer stream;
(d) routing the warm beer stream to a baffled flash tank, wherein the baffled flash tank is configured to separate the warm beer stream into a first vapor stream and liquid beer;
(e) routing the liquid beer into a beer distillation column, wherein the beer distillation column is configured to separate the liquid beer into a second vapor stream and solid precipitates; and
(f) discharging the first vapor stream from the baffled flash tank into a beer distillation column vapor discharge line.

2. The method of claim 1, further comprising discharging the second vapor stream from the beer distillation column by the beer distillation column vapor discharge line.

3. The method of claim 2, further comprising combining the second vapor stream with the first vapor stream.

4. The method of claim 1, prior to the routing in step (c), further comprising pressurizing the cold beer stream.

5. The method of claim 3, wherein the cold beer stream is pressurized to a range of about 55 psia to about 115 psia.

6. The method of claim 1, wherein the warm mash has a temperature in a range of about 180° F. to about 200° F.

7. The method of claim 1, wherein the cold mash has a temperature in a range of about 110° F. to about 130° F.

8. The method of claim 1, wherein the cold beer stream has a temperature in a range of about 80° F. to about 95° F.

9. The method of claim 1, wherein the cold beer stream has an ethanol concentration in a range of about 5% to about 30% v/v.

10. The method of claim 1, wherein the warm beer stream has a temperature in a range of about 155° F. to about 170° F.

11. The method of claim 1, wherein the warm beer stream has an ethanol concentration in a range of about 5% to about 30% v/v.

12. The method of claim 1, wherein the liquid beer has a temperature in a range of about 150° F. to about 160° F.

13. The method of claim 1, wherein the liquid beer has an ethanol concentration in a range of about 5% to about 30% v/v.

14. The method of claim 1, wherein the first vapor stream has an ethanol concentration in a range of about 50% to about 60% v/v.

15. The method of claim 1, wherein the second vapor stream has an ethanol concentration in a range of about 50% to about 60% v/v.

16. The method of claim 1, wherein the baffled flash tank is coupled to the beer distillation column by two outlets such that pressure and temperature of the baffled flash tank and the beer distillation column are substantially equivalent;
wherein a first outlet is configured to route the first vapor stream from the baffled flash tank to the beer distillation column vapor discharge line; and
wherein a second outlet is configured to route the liquid beer from the baffled flash tank to the beer distillation column.

17. The method of claim 16, wherein the pressure of the baffled flash tank and the beer distillation column is in a range of about 3 psia to about 20 psia.

18. The method of claim 16, wherein the temperature of the baffled flash tank and top of the beer distillation column is in a range of about 155° F. to about 165° F.

19. A system for a dry mill ethanol plant comprising:
a heat recovery system coupled to a baffled flash tank, wherein the baffled flash tank is configured to receive warm beer stream; and
a distillation system coupled to the baffled flash tank, wherein the baffled flash tank is configured to separate the warm beer stream into a vapor stream and liquid beer.

20. The system of claim 19, wherein the baffled flash tank comprises two outlets, a first outlet coupled to a beer distillation column vapor discharge line for routing the vapor stream from the baffled flash tank; and a second outlet coupled to a beer distillation column inlet for routing the liquid beer from the baffled flash tank.
